SEQUENCE LISTING (16 sequences)

SEQ ID NO: 1 - protein, 247 aa, Pseudomonas saitens
  misc_feature (1)..(247): KCTC 13107BP
MEKVKAILFDKDGTLMDFHSIWIKVAEELVAECISLYHLPSTIGQTLLEEIGVEGAFVNPRSAIAAGTSLDVAKGLCNYIESAREEEMHQWVSEKLFSLMYEHRSHMKMTADLPKVLQALKDKGFILGVVTADDFAPTELFLKQYKLENFFDYIIASDTFPAQKPDKKIIEVFCEKFNLESCEVAVVGDTPTDLHLAKNGDCYAIGVLSGTGDRPTLEPLADLVLDSVGEFISQSGEFFWEKEKSNV

SEQ ID NO: 2 - DNA, 744 bp, Pseudomonas saitens
  misc_feature (1)..(744): KCTC 13107BP
atggaaaaagttaaagcgatcctgttcgataaagatggcaccctgatggatttccacagcatctggatcaaagttgcggaagaactggttgcggaatgcatcagcctgtaccacctgccgtccaccatcggtcagaccctgctggaagaaatcggcgttgaaggcgcgttcgttaacccgcgtagcgcgatcgcggcgggcaccagcctggatgttgcgaaaggcctgtgcaactacatcgaaagcgcgcgtgaagaagaaatgcaccagtgggttagcgaaaaactgttcagcctgatgtacgaacaccgtagccacatgaaaatgaccgcggatctgccgaaagttctgcaggctctgaaagataaaggtttcatcctgggcgttgttaccgcggatgatttcgcgccgaccgaactgttcctgaaacagtacaaactggaaaacttcttcgattacatcatcgcgagcgataccttcccggcacagaaaccggataagaaaatcatcgaagtgttctgcgaaaaattcaacctggaaagctgtgaagttgcggttgttggcgataccccgaccgatctgcacctggcgaaaaacggtgattgctacgcgatcggtgttctgtccggtaccggtgatcgtccgaccctggaaccgctggcggatctggttctggattctgttggtgaattcatcagccagagcggtgaattcttctgggaaaaagaaaaaagcaacgtttaa

SEQ ID NO: 3 - DNA, 43 bp, Artificial Sequence
  Synthetic primer
agaaggagatataccatggaaaaagttaaagcgatcctgttcg

SEQ ID NO: 4 - DNA, 53 bp, Artificial Sequence
  Synthetic primer
tcctttcgggctttgttaaacgttgcttttttctttttcccagaagaattcac

SEQ ID NO: 5 - DNA, 33 bp, Artificial Sequence
  Synthetic primer
ggcaccctgatggatgaccacagcatctggatc

SEQ ID NO: 6 - DNA, 33 bp, Artificial Sequence
  Synthetic primer
gatccagatgctgtggtcatccatcagggtgcc

SEQ ID NO: 7 - DNA, 33 bp, Artificial Sequence
  Synthetic primer
ggcaccctgatggatagccacagcatctggatc

SEQ ID NO: 8 - DNA, 33 bp, Artificial Sequence
  Synthetic primer
gatccagatgctgtggctatccatcagggtgcc

SEQ ID NO: 9 - DNA, 33 bp, Artificial Sequence
  Synthetic primer
ggcaccctgatggatgtacacagcatctggatc

SEQ ID NO: 10 - DNA, 33 bp, Artificial Sequence
  Synthetic primer
gatccagatgctgtgtacatccatcagggtgcc

SEQ ID NO: 11 - DNA, 744 bp, Artificial Sequence
  Synthetic Hdl4a mutant
atggaaaaagttaaagcgatcctgttcgataaagatggcaccctgatggatgaccacagcatctggatcaaagttgcggaagaactggttgcggaatgcatcagcctgtaccacctgccgtccaccatcggtcagaccctgctggaagaaatcggcgttgaaggcgcgttcgttaacccgcgtagcgcgatcgcggcgggcaccagcctggatgttgcgaaaggcctgtgcaactacatcgaaagcgcgcgtgaagaagaaatgcaccagtgggttagcgaaaaactgttcagcctgatgtacgaacaccgtagccacatgaaaatgaccgcggatctgccgaaagttctgcaggctctgaaagataaaggtttcatcctgggcgttgttaccgcggatgatttcgcgccgaccgaactgttcctgaaacagtacaaactggaaaacttcttcgattacatcatcgcgagcgataccttcccggcacagaaaccggataagaaaatcatcgaagtgttctgcgaaaaattcaacctggaaagctgtgaagttgcggttgttggcgataccccgaccgatctgcacctggcgaaaaacggtgattgctacgcgatcggtgttctgtccggtaccggtgatcgtccgaccctggaaccgctggcggatctggttctggattctgttggtgaattcatcagccagagcggtgaattcttctgggaaaaagaaaaaagcaacgtttaa

SEQ ID NO: 12 - DNA, 744 bp, Artificial Sequence
  Synthetic Hdl4a mutant
atggaaaaagttaaagcgatcctgttcgataaagatggcaccctgatggatagccacagcatctggatcaaagttgcggaagaactggttgcggaatgcatcagcctgtaccacctgccgtccaccatcggtcagaccctgctggaagaaatcggcgttgaaggcgcgttcgttaacccgcgtagcgcgatcgcggcgggcaccagcctggatgttgcgaaaggcctgtgcaactacatcgaaagcgcgcgtgaagaagaaatgcaccagtgggttagcgaaaaactgttcagcctgatgtacgaacaccgtagccacatgaaaatgaccgcggatctgccgaaagttctgcaggctctgaaagataaaggtttcatcctgggcgttgttaccgcggatgatttcgcgccgaccgaactgttcctgaaacagtacaaactggaaaacttcttcgattacatcatcgcgagcgataccttcccggcacagaaaccggataagaaaatcatcgaagtgttctgcgaaaaattcaacctggaaagctgtgaagttgcggttgttggcgataccccgaccgatctgcacctggcgaaaaacggtgattgctacgcgatcggtgttctgtccggtaccggtgatcgtccgaccctggaaccgctggcggatctggttctggattctgttggtgaattcatcagccagagcggtgaattcttctgggaaaaagaaaaaagcaacgtttaa

SEQ ID NO: 13 - DNA, 744 bp, Artificial Sequence
  Syntethic Hdl4a mutant
atggaaaaagttaaagcgatcctgttcgataaagatggcaccctgatggatgtacacagcatctggatcaaagttgcggaagaactggttgcggaatgcatcagcctgtaccacctgccgtccaccatcggtcagaccctgctggaagaaatcggcgttgaaggcgcgttcgttaacccgcgtagcgcgatcgcggcgggcaccagcctggatgttgcgaaaggcctgtgcaactacatcgaaagcgcgcgtgaagaagaaatgcaccagtgggttagcgaaaaactgttcagcctgatgtacgaacaccgtagccacatgaaaatgaccgcggatctgccgaaagttctgcaggctctgaaagataaaggtttcatcctgggcgttgttaccgcggatgatttcgcgccgaccgaactgttcctgaaacagtacaaactggaaaacttcttcgattacatcatcgcgagcgataccttcccggcacagaaaccggataagaaaatcatcgaagtgttctgcgaaaaattcaacctggaaagctgtgaagttgcggttgttggcgataccccgaccgatctgcacctggcgaaaaacggtgattgctacgcgatcggtgttctgtccggtaccggtgatcgtccgaccctggaaccgctggcggatctggttctggattctgttggtgaattcatcagccagagcggtgaattcttctgggaaaaagaaaaaagcaacgtttaa

SEQ ID NO: 14 - protein, 247 aa, Artificial Sequence
  Synthetic F18D mutant
MEKVKAILFDKDGTLMDDHSIWIKVAEELVAECISLYHLPSTIGQTLLEEIGVEGAFVNPRSAIAAGTSLDVAKGLCNYIESAREEEMHQWVSEKLFSLMYEHRSHMKMTADLPKVLQALKDKGFILGVVTADDFAPTELFLKQYKLENFFDYIIASDTFPAQKPDKKIIEVFCEKFNLESCEVAVVGDTPTDLHLAKNGDCYAIGVLSGTGDRPTLEPLADLVLDSVGEFISQSGEFFWEKEKSNV

SEQ ID NO: 15 - protein, 247 aa, Artificial Sequence
  Synthetic F18S mutant
MEKVKAILFDKDGTLMDSHSIWIKVAEELVAECISLYHLPSTIGQTLLEEIGVEGAFVNPRSAIAAGTSLDVAKGLCNYIESAREEEMHQWVSEKLFSLMYEHRSHMKMTADLPKVLQALKDKGFILGVVTADDFAPTELFLKQYKLENFFDYIIASDTFPAQKPDKKIIEVFCEKFNLESCEVAVVGDTPTDLHLAKNGDCYAIGVLSGTGDRPTLEPLADLVLDSVGEFISQSGEFFWEKEKSNV

SEQ ID NO: 16 - protein, 247 aa, Artificial Sequence
  Synthetic F18V mutant
MEKVKAILFDKDGTLMDVHSIWIKVAEELVAECISLYHLPSTIGQTLLEEIGVEGAFVNPRSAIAAGTSLDVAKGLCNYIESAREEEMHQWVSEKLFSLMYEHRSHMKMTADLPKVLQALKDKGFILGVVTADDFAPTELFLKQYKLENFFDYIIASDTFPAQKPDKKIIEVFCEKFNLESCEVAVVGDTPTDLHLAKNGDCYAIGVLSGTGDRPTLEPLADLVLDSVGEFISQSGEFFWEKEKSNV

(19) United States
(12) Patent Application Publication
(10) Pub. No.: US 2019/0078067 A1
(43) Pub. Date: Mar. 14, 2019

(54) HALOACID DEHALOGENASE hdl4a PROTEIN VARIANT AND METHOD OF REDUCING CONCENTRATION OF FLUORINE-CONTAINING COMPOUND IN A SAMPLE USING THE SAME

(71) Applicant: Samsung Electronics Co., Ltd., Suwon-si (KR)

(72) Inventors: Jinha Kim, Hwaseong-si (KR); Taeyong Kim, Daejeon (KR); Jinhwan Park, Suwon-si (KR); Seunghoon Song, Suwon-si (KR)

(21) Appl. No.: 16/107,635

(22) Filed: Aug. 21, 2018

(30) Foreign Application Priority Data
Sep. 13, 2017 (KR) 10-2017-0117233

(51) Int. Cl. C12N 9/14; B01D 53/84

(52) U.S. Cl. CPC C12N 9/14; B01D 2255/804; B01D 2251/95; B01D 53/84

(57) ABSTRACT

Provided are a protein variant of haloacid dehalogenase hdl4a and a method of reducing a concentration of a fluorine-containing compound in a sample using the protein variant.

### CROSS-REFERENCE TO RELATED APPLICATION

This application claims the benefit of Korean Patent Application No. 10-2017-0117233, filed on Sep. 13, 2017, in the Korean Intellectual Property Office, the disclosure of which is incorporated herein in its entirety by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 14,833 Byte ASCII (Text) file named “739006_ST25.TXT,” created on May 11, 2018.

### BACKGROUND

### 1. Field

The present disclosure relates to a recombinant microorganism, which includes a foreign gene encoding haloacid dehalogenase hdl4a protein or a variant thereof, a composition including a foreign gene encoding haloacid dehalogenase hdl4a protein or a variant thereof for use in removing a fluorine-compound in a sample, and a method of reducing a concentration of a fluorine-compound in a sample using the protein or the variant thereof.

### 2. Description of the Related Art

The emissions of greenhouse gases which have accelerated global warming are serious environmental problems, and regulations to reduce and prevent the emissions of greenhouse gases have been tightened. Among the greenhouse gases, fluorinated gases (F-gases), such as perfluorocarbons (PFCs), hydrofluorocarbons (HFCs), and sulfur hexafluoride (SF₆), show low absolute emission, but have a long half-life and a very high global warming potential, resulting in significantly adverse environmental impacts. The amount of F-gases emitted from semiconductor and electronics industries, which are major causes of F-gas emission, has exceeded the assigned amount of greenhouse gas emissions and continues to increase. Therefore, costs required for decomposition of greenhouse gases and greenhouse gas emission allowances are increasing every year.

Pyrolysis or catalytic thermal oxidation processes have been used in the decomposition of F-gases. However, such processes have the disadvantages of a limited decomposition rate, emission of secondary pollutants, high cost, etc. However, biological decomposition of F-gases using a microbial biocatalyst would allow F-gases to be treated in a more economical and environmentally-friendly manner.

Therefore, there is a need to develop new microorganisms and methods for the biological decomposition of F-gases. This invention provides such microorganisms and methods.

### SUMMARY

Provided is a recombinant microorganism including a foreign gene encoding a haloacid dehalogenase hdl4a protein or a variant thereof.

Also provided is a composition for use in reducing a fluorine-containing compound in a sample, the composition including a haloacid dehalogenase hdl4a protein or a variant thereof.

Further provided is a method of reducing a concentration of a fluorine-containing compound in a sample, the method including contacting a haloacid dehalogenase hdl4a of a variant thereof with a sample including a fluorine-containing compound, so as to reduce the concentration of the fluorine-containing compound in the sample.

Also provided is a variant of a haloacid dehalogenase hdl4a protein and a polynucleotide encoding the variant.

### BRIEF DESCRIPTION OF THE DRAWINGS

These and/or other aspects will become apparent and more readily appreciated from the following description of the embodiments, taken in conjunction with the accompanying drawings in which:

FIG. 1 is a vector map of a pET28a-Hdl4a vector.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

The term “gene” as used herein refers to a polynucleotide that expresses a particular protein. A gene may include regulatory sequences. Examples of the regulatory sequences include a 5′-non coding sequence and a 3′-non coding sequence. The regulatory sequence may include a promoter, an enhancer, an operator, a ribosome binding site, a polyA binding site, a terminator region, and the like.

The term “sequence identity” with respect to a nucleic acid or a polypeptide refers to a degree of identity of bases or amino acid residues in sequences in a comparative region after aligning two sequences to best match. The sequence identity is a value measured by comparing two sequences in a certain comparative region through optimal alignment of the two sequences, wherein some portions of the sequences in the comparative region may be added or deleted compared to a reference sequence. A percentage of sequence identity may be for example, calculated as follows: two sequences that are optimally aligned are compared in the entire comparative region; the number of locations where the same amino acids or nucleic acids appear in both sequences is determined to the number of matching locations; the number of matching locations is divided by the total number of locations (i.e., the size of a range) in the comparative region; and the result of the division is multiplied by 100 to obtain the percentage of the sequence identity. The percentage of the sequence identity may be determined using a known sequence comparison program, such as BLASTN or BLASTP (NCBI), CLC Main Workbench (CLC bio), or MegAlign™ (DNASTAR Inc). Unless otherwise mentioned in the specification, the selection of the parameters used to execute the program may be as follows: E-value=0.00001 and H-value=0.001.

An aspect of the disclosure provides a recombinant microorganism including a foreign gene encoding a haloacid dehalogenase (HAD) hdl4a protein or a variant thereof.

Regarding the recombinant microorganism, the HAD hdl4a may be an enzyme derived from Pseudomonas saitens (KCTC 13107BP). The strain was separated from sludge of the wastewater discharged from a semiconductor plant, and is capable of reducing a concentration of CF₄ in a sample. The strain was deposited at the Korean Collection for Type Culture (KCTC), which is an international depository authority under the Budapest Treaty, on Sep. 12, 2016, and assigned the accession number.

The enzyme may be haloacid dehalogenase belonging to EC 3.8.1.2.

The protein or the variant thereof may have 85% or more, 90% or more, 95% or more, 97% or more, 98% or more, 99% or more, or 100% or more sequence identity with an amino acid sequence of SEQ ID NO: 1.

The variant may have an amino acid alteration at a position corresponding to F18 of SEQ ID NO: 1. The variant may have an activity of an enzyme belonging to the HAD, such as haloacid dehalogenase belonging to EC 3.8.1.2. The variant may be provided by substituting a residue at position F18 of hdl4a having the amino acid sequence of SEQ ID NO: 1 or corresponding position of an hdl4a protein with a different amino acid sequence (e.g., an amino acid sequence with 85% or more, 90% or more, 95% or more, 97% or more, 98% or more, 99% or more sequence identity to SEQ ID NO: 1) with a different amino acid, such as one of the 19 other natural amino acids. The amino acid alteration may include substitution of D, S, or V for F18, or a combination thereof. Alternatively, the alteration may be a conservative substitution for D, S, or V at position F18. In other words, the alteration may be a substitution of the amino acid corresponding to F18 with an amino acid that is conservative with respect to D, S, or V. A conservative substitution for D at position 18 may be F18E. A conservative substitution for S at position 18 may be F18T, F18C, F18Y, F18N, or F18Q. A conservative substitution for V at position 18 may be F18G, F18A, F18V, F18L, F18I, F18M, F18W, or F18P. The protein may have 85% or more, 90% or more, 95% or more, 97% or more, 98% or more, 99% or more, or 100% or more sequence identity with the amino acid sequence of SEQ ID NO: 1.

The amino acid alteration may include substitution, insertion, or deletion. The substitution may include substitution with an amino acid that is modified after translation. The substitution may include substitution with one of 20 natural amino acids other than the amino acid corresponding to F18 of the hdl4a sequence being modified. Amino acids used herein and abbreviations thereof are shown in Table 1.

TABLE 1

| | |
|---|---|
| Abbreviation | Amino acid |

| | | |
|---|---|---|
| A | Ala | Alanine |
| C | Cys | Cysteine |
| D | Asp | Aspartic acid |
| E | Glu | Glutamic acid |
| F | Phe | Phenylalamine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

The substitution of the residue corresponding to position F18 of SEQ ID NO: 1 may be a conservative substitution. The term “conservative mutation” or “conservative substitution” as used herein, respectively, refers to an amino acid mutation that one of ordinary skill in the art would consider a conservative to a first mutation. The term “conservative” as used herein means a similar amino acid in terms of the amino acid characteristics. For example, when a non-aliphatic amino acid residue (e.g., Ser) at a specific position is substituted with an aliphatic amino acid residue (e.g., Leu), a substitution with a different aliphatic amino acid (e.g., ILe or Val) at the same position is referred to as a conservative mutation. In addition, the amino acid characteristics include size of the residue, hydrophobicity, polarity, charge, pK-value, and other amino acid characteristics known in the art. Accordingly, a conservative mutation may include substitution, such as basic for basic, acid for acid, polar for polar, and the like. Conservative substitutions may be made, for example, according to Table 2 below which describes a generally accepted grouping of amino acid characteristics.

TABLE 2

| Set | Amino acids |
|---|---|
| Non-polar | G A V L I M F W P |
| Polar | S T C Y N Q |
| Acidic | D E |
| Basic | K R H |

The term “corresponding” as used herein refers to the amino acid position of a protein of interest that aligns with the mentioned position of a reference protein (e.g., position F18 of SEQ ID NO: 1) when amino acid sequences of the protein of interest and the reference protein are aligned using an art-acceptable protein alignment program, such as the BLAST pairwise alignment or the well known Lipman-Pearson Protein Alignment program. For example, the amino acid residue to be altered in a protein of interest may be an amino acid residue which corresponds, as determined by the alignment methods described herein or otherwise known in the art, to the amino acid residue of position F18 of amino acid sequence of SEQ ID NO: 1. The protein of interest may be HAD, which belongs to, for example, EC 3.8.1.2. The database (DB) in which the reference sequence is stored may be Reference Sequence (RefSeq) non-redundant protein database of NCBI. The parameters used for the sequence alignment may be as follows: E-value 0.00001 and H-value 0.001.

The recombinant microorganism may be bacteria or fungi, and the bacteria may be Gram positive or Gram-negative. The Gram-negative bacteria may belong to the Enterobacteriaceae family. The Gram-negative bacteria may belong to the genus Escherichia, the genus Salmonella, the genus Xanthobacter, or the genus Pseudomonas. The microorganism belonging to the genus Escherichia may be E. coli. The microorganism belonging to the genus Xanthobacter may be X. autotrophicus. The Gram-positive bacteria may belong to the genus Corynebacterium or the genus Bacillus.

The recombinant microorganism may comprise a foreign (heterologous) nucleic acid encoding the hld4a protein or variant thereof. For example, the recombinant microorganism may comprise at least one polynucleotide having a nucleotide sequence of SEQ ID NO: 2.

Another aspect of the disclosure provides a composition including a haloacid dehalogenase hdl4a protein or a variant thereof, for use in removing a fluorine-containing compound in a sample. Unless otherwise mentioned in the specification, the recombinant protein or the variant thereof is the same as described above. The fluorine-containing compound may be represented by Formula 1 or 2:

C(R¹)(R²)(R³)(R⁴) <Formula 1>

(R⁵)(R⁶)(R⁷)C—[C(R¹¹)(R¹²)]n-C(R⁸)(R⁹)(R¹⁰), <Formula 2>

In Formulae 1 and 2, n may be an integer from 0 to 10, R¹, R², R³, and R⁴ may each independently be fluorine (F), chlorine (CI), bromine (Br), iodine (I), or hydrogen (H), provided at least one selected from R¹, R², R³, and R⁴ is F; R⁵, R⁶, R⁷, R⁸, R⁹, R¹⁰, R¹¹, and R¹² may each be independently F, Cl, Br, I, or H, provided at least one selected from R⁵, R⁶, R⁷, R⁸, R⁹, R¹⁰, R¹¹, and R¹² is F.

For example, the fluorine-containing compound may be, for example, CHF₃, CH₂F2, CH₃F, or CF₄. The term “removal” as used herein refers to any reduction of the concentration of the fluorine-containing compound in the sample, including complete removal of the fluorine-containing compound from the sample.

In the composition, the hld4a protein or the variant thereof may be included in a recombinant microorganism including a foreign gene that encodes the protein or the variant thereof. The composition may include the recombinant microorganism itself, a lysate thereof, or a water soluble material fraction of the lysate. Unless otherwise mentioned in the specification, the recombinant microorganism is the same as described above.

Removal of the fluorine-containing compound may include reduction of the concentration of the fluorine-containing compound achieved by cleavage of a C—F bond of the fluorine-containing compound, conversion of the fluorine-containing compound into a different material, or accumulation of the fluorine-containing compound in a cell. The conversion of the fluorine-containing compound may include introduction of a hydrophilic group, such as a hydroxyl group, to the fluorine-containing compound, or introduction of a carbon-carbon double bond or a carbon-carbon triple bond to the fluorine-containing compound.

The sample containing the fluorine-containing compound may be a liquid sample or a gaseous sample. The sample may be, for instance, industrial sewage or waste gas.

Another aspect of the disclosure provides a method of reducing a concentration of a fluorine-containing compound in a sample, the method including contacting a haloacid dehalogenase hdl4a protein or a variant thereof with a sample including a fluorine-containing compound represented by Formula 1 or 2, so as to reduce the concentration of the fluorine-containing compound in the sample. Unless otherwise mentioned in the specification, the recombinant protein or the variant thereof is the same as described above.

The contacting of the hld4a protein or variant thereof with the sample may be performed in an air-tight closed container. The contacting may include gas-liquid contacting of a gaseous sample with a liquid containing the variant of the hdl4a protein or variant thereof. In addition, the contacting may be liquid-liquid contacting of a liquid sample with a liquid containing the variant of the hdl4a protein or variant thereof. The liquid-liquid contacting may include mixing.

The protein or the variant thereof may be included in a recombinant microorganism including a foreign gene that encodes the protein or the variant thereof. In this regard, the contacting of the hld4a protein or variant thereof with the sample may include contacting the sample with a cell expressing the protein; thus, the sample may contact the cell first, and then contact the protein or the variant thereof in the cell. The protein or the variant thereof may be included in the recombinant microorganism, a lysate thereof, or a water soluble material fraction of the lysate. The foreign gene encoding the hld4a protein or variant thereof included in the recombinant microorganism is the same as described above.

Regarding the method, the contacting may be performed under conditions where the recombinant microorganism may survive in an air-tight closed container. Such conditions for the survival of the recombinant microorganism may include conditions where the recombinant microorganism may proliferate or conditions where the recombinant microorganism may be allowed to be in a resting state. In this regard, the contacting may include culturing a microorganism in the presence of the sample containing the fluorine-containing compound. The culturing may be performed under aerobic or anaerobic conditions.

Regarding the method, the sample may be a liquid sample or a gaseous sample. The sample may be industrial sewage or waste gas.

Another aspect of an embodiment provides a variant of a haloacid dehalogenase hdl4a protein, wherein the variant includes an amino acid alteration in an amino acid residue corresponding to position F18 of SEQ ID NO: 1. The variant is the same as described above with respect to the recombinant microorganism and other aspects of the disclosure.

Another aspect of the disclosure provides a polynucleotide encoding a variant of a haloacid dehalogenase hdl4a protein, wherein the variant includes amino acid alteration at an amino acid residues corresponding to position F18 of SEQ ID NO: 1. The variant is the same as described above with respect to the recombinant microorganism and other aspects of the disclosure.

The polypeptide encoding the variant may be included in a vector. For use as a vector, any vehicle that can be used to introduce a polynucleotide to a microorganism may be used. The vector may be, for instance, a plasmid vector or a viral vector.

Another aspect of the disclosure provides a method of preparing a microorganism having increased ability to remove a fluorine-containing compound in a sample. The method includes introducing a gene that encodes haloacid dehalogenase hdl4a protein or a variant thereof to a microorganism. The hdl4a protein or a variant thereof is the same as described above with respect to the recombinant microorganism and other aspects of the disclosure.

According to an aspect of another embodiment, the recombinant microorganism may be used for removing the fluorine-containing compound in the sample.

According to an aspect of another embodiment, the composition including the protein or the variant thereof may be used for removing the fluorine-containing compound in the sample.

According to an aspect of another embodiment, the method of reducing the concentration of the fluorine-containing compound in the sample may effectively reduce the concentration of the fluorine-containing compound in the sample.

According to an aspect of another embodiment, the protein or the variant thereof and the polynucleotide encoding the protein or the variant thereof same may be used to remove the fluorine-containing compound in the sample or to produce the variant.

Hereinafter, the present invention will be described in more detail with reference to Examples. However, these Examples are provided for illustrative purposes only, and the invention is not intended to be limited by these Examples.

### Example 1: Recombinant E. coli Expressing Hdl4a Gene and Removal of a Fluorine-Containing Compound in a Sample Using the Recombinant E. coli

Recombinant E. coli expressing an HAD gene, such as Hdl4a gene derived from P. saitens KCTC 13107BP, or a gene of a variant of the Hdl4a was prepared, and the effect on the removal of CF₄ in a sample was confirmed by using recombinant E. coli.

1. Amplification of a Haloacid Dehalogenase Gene (Hdl4) Derived from P. Saitens KCTC 13107BP and Introduction of the Gene to E. coli

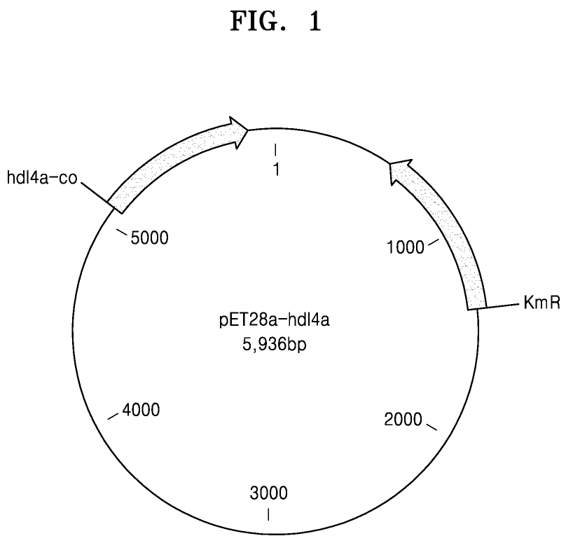

A sequence (SEQ ID NO: 2) of the Hdl4a gene derived from P. saitens KCTC 13107BP was amplified. For the amplification, PCR was performed using the genome DNA of the strain as a template and a set of primers having nucleotide sequences of SEQ ID NOs: 3 and 4. The amplified genes were ligated with a pET28a (Novagen, Cat. No. 69864), which was digested with restriction enzymes, NcoI and HindIII, using the InFusion Cloning Kit (Clontech Laboratories, Inc.), thereby preparing a pET28a-Hdl4a vector. FIG. 1 is a vector map of the pET28a-Hdl4a. A Hdl4a protein has an amino acid sequence of SEQ ID NO: 1, and a gene thereof has a nucleotide sequence of SEQ ID NO: 2.

Next, the prepared pET28a-Hdl4a vector was introduced to E. coli BL21 by a heat shock method, and then, cultured in an LB plate containing 50 μg/mL of kanamycin. Strains showing kanamycin resistance were selected. Then, a finally selected strain was designated as a recombinant E. coli BL21/pET28a-Hdl4awt.

2. Recombinant E. coli Expressing a Variant of the Hdl4a Gene

A variant was prepared to improve the activity of the Hdl4a gene on the removal of a fluorine-containing compound in a sample. Phenylalanine at position 18 (hereinafter, referred to as “F18”) of the Hdl4a protein comprising the amino acid sequence of SEQ ID NO: 1 was substituted with each of other 19 natural amino acids. The substitution may be represented by “F18X” (wherein X indicates natural amino acids other than phenylalanine). The effect of E. coli, which was prepared by introducing a gene encoding the prepared variant thereto, on the removal of CF₄ in a sample was confirmed.

The preparation of the F18X variant of SEQ ID NO: 1 was achieved by using the QuikChange II Site-Directed Mutagenesis Kit (Agilent Technology, USA). Site-directed mutagenesis using the kit was performed by using PfuUltra high-fidelity (HF) DNA polymerase for mutagenic primer-directed replication of two plasmid strands with the highest fidelity. The basic procedure utilizes a super-coiled double-stranded DNA (dsDNA) vector with an insert of interest and two synthetic oligonucleotide primers, both containing the desired mutation. The oligonucleotide primers, each complementary to opposite strands of the vector, were extended during temperature cycling by PfuUltra HF DNA polymerase, without primer displacement. Extension of the oligonucleotide primers generated a mutated plasmid containing staggered nicks. Following temperature cycling, the product was treated with DpnI. The DpnI endonuclease (target sequence: 5′-Gm⁶ATC-3′) was specific for methylated and hemimethylated DNA, and was used to digest the parental DNA template for the selection of mutation-containing synthesized DNA. Afterwards, the nicked vector DNA incorporating the desired mutations was then transformed into XL1-Blue supercompetent cells.

Among the primer sets used to induce mutagenesis of F18X, primer sets of SE ID NOs: 5 and 6, primer sets of SEQ ID NOs: 7 and 8, and primer sets of SEQ ID NOs: 9 and 10 were used for the F18D, F185, and F18V variant gene. The Hdl4a proteins having the F18D, F185, and F18V variant may each be encoded by a nucleotide sequence of SEQ ID NOs: 11, 12, and 13 and have the amino acid sequence of SEQ ID NO: 14, 15, and 16, respectively.

In detail, PCR was performed by using the pET28a-Hdl4awt vector prepared in section (1) as a template and the primer sets for each of the variants as a primer, and a PfuUltra HF DNA polymerase to obtain variant vectors including staggered nicks. These vector products were treated with DpnI to select variant-containing synthesized DNA. Afterwards, the nicked vector DNA incorporating a desired variant was then transformed into XL1-Blue supercompetent cells, thereby cloning the pET28a-Hdl4amt vector.

Lastly, the cloned pET28a-Hdl4awt vector was introduced to a strain of E. coli BL21 in the same manner as in section (1), and a finally selected strain was designated as a recombinant E. coli BL21/pET28a-Hdl4amt.

3. Effect of Recombinant E. coli Including a Hdl4a Gene and a Variant Thereof Introduced Thereto on the Removal of CF₄ in a Sample

The effect of the E. coli BL21/pET28a-Hdl4awt and the E. coli BL21/pET28a-Hdl4amt prepared in sections (1) and (2) and including the Hdl4a gene and the variant thereof introduced thereto on the removal of CF₄ in a sample was confirmed.

A strain of the E. coli BL21/pET28a-Hdl4awt and a strain of the E. coli BL21/pET28a-Hdl4amt were cultured in a LB medium with stirring at a temperature of 37□ at a speed of 250 rpm, and at an OD₆₀₀ of about 0.5, IPTG 0.2 mM was added to the medium, followed by being cultured overnight with stirring at a temperature of 30□ at a speed of 250 rpm. Then, the cells were harvested and suspended in a LB medium, so as to have a cell concentration OD₆₀₀ of 1.0. 10 mL of the cell solution was added to a 60 mL serum bottle, and the serum bottle was air tightly sealed. The LB medium was supplemented with 10 g of tripton per 1 L of distilled water, 5 g of an enzyme extract, and 10 g of NaCl. Next, CF₄ in a gas phase was injected to the serum bottle through a rubber stopper of a cap of the serum bottle by using a syringe, so as to have 1,000 ppm of CF₄ in a head space of the serum bottle. Afterwards, the serum bottle was cultured for 4 days with stirring at a temperature of 30□ at a speed of 230 rpm. The experiments were performed in triplicate. After incubation, 0.5 mL of CF₄ gas was collected by using a 1.0 mL syringe from the head space, which did not contain the medium, of the serum bottle, and then, was injected into a gas chromatograph (GC) column (Agilent 7890, Palo Alto, Calif., USA). The injected CF₄ gas was separated by a CP-PoraBOND Q column (25 m length, 0.32 mm inner diameter, 5 um film thickness, Agilent), and changes in the concentration of CF₄ gas was analyzed by mass spectrometry (MS) (Agilent 5973, Palo Alto, Calif., USA). Here, helium was used as a carrier gas, and was flowed into the column at a rate of 1.5 ml/min. Regarding conditions for the GC, a temperature at an inlet was 250□, and an initial temperature was maintained at 40□ for 2 minutes and raised up to 290□ at a speed of 20□/min. Regarding conditions for the MS, an ionization energy was 70 eV, an interface temperature was 280□, an ion source temperature was 230□, and a quadrupole temperature was 150□. As a result, in Table 3, strains including the variant showed the activity of removing CF₄ gas in a sample as compared to a wild type.

TABLE 3

| | | Decomposition rate of CF₄ (%, as |
|---|---|---|
| Strain | Residual CF₄ (%) | compared to a control group) |
| Control group | 100 | — |
| Wild type hdl4a | 97.2 | 2.8 |
| F18D | 96.1 | 3.9 |
| F18S | 94.3 | 5.7 |
| F18V | 94.7 | 5.3 |

In Table 3, the control group was E. coli BL21 to which an empty pET28a vector was introduced instead of the pET-BC3334mt vector, and the wild type was a strain to which BL21/pET28a-Hdl4awt was introduced. F18D, F18S, and F18V were respectively BL21/pET28a-Hdl4amt having the F18D, F18S, or F18V variants.

As shown in Table 3, the E. coli including the wild type gene showed a reduction of CF₄ by 2.8% as compared to the control group, wherein the E. coli including the variants of the Hdl4a gene, i.e., the F18D, F18S, and F18V variants, showed a reduction of CF₄ by 3.9%, 5.7%, and 5.3%, respectively, as compared to the control group.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism comprising a heterologous haloacid dehalogenase hdl4a protein, or a variant haloacid dehalogenase hdl4a protein comprising an amino acid alteration in an amino acid residue corresponding to position F18 of SEQ ID NO: 1.

2. The microorganism of claim 1, wherein the amino acid alteration comprises substitution of D, S, or V or substitution of a different amino acid for F18 that is conservative with respect to D, S, or V, wherein the substitution of a different amino acid for F18 that is conservative with respect to D is F18E, the substitution of a different amino acid for F18 that is conservative with respect to S is F18T, F18C, F18Y, F18N, or F18Q, and the substitution of a different amino acid for F18 that is conservative with respect to V is F18G, F18A, F18V, F18L, F18I, F18M, F18W, or F18P.

3. The microorganism of claim 1, wherein the hdl4a protein or variant hdl4a protein has 85% or more sequence identity with of SEQ ID NO: 1.

4. A composition comprising
(a) an isolated haloacid dehalogenase hdl4a protein or a variant hdl4a protein or a recombinant microorganism expressing a heterologous hdl4a protein or a variant hdl4a protein, wherein the variant hdl4a protein comprises an amino acid alteration in an amino acid residue corresponding to position F18 of SEQ ID NO: 1;
and (b) a fluorine-containing compound represented by Formula 1 or 2:
C(R¹)(R²)(R³)(R⁴) <Formula 1>
(R⁵)(R⁶)(R⁷)C—[c(R¹¹)(R¹²)]n-C(R⁸)(R⁹)(R¹⁰), <Formula 2>
wherein, in Formula 1 and 2:
n is an integer from 0 to 10,
R¹, R², R³, and R⁴ are each independently fluorine (F), chlorine (Cl), bromine (Br), iodine (I), or hydrogen (H), wherein at least one of R¹, R², R³, or R⁴ is F; and
and R⁵, R⁶, R⁷, R⁸, R⁹, R¹⁰, R¹¹, and R¹² are each independently F, Cl, Br, I, or H, wherein at least one of R⁵, R⁶, R⁷, R⁸, R⁹, R¹⁰, R¹¹, or R¹² is F.

5. The composition of claim 4, wherein the amino acid alteration comprises substitution of D, S, or V or substitution of a different amino acid for F18 that is conservative with respect to D, S, or V, wherein the substitution of a different amino acid for F18 that is conservative with respect to D is F18E, the substitution of a different amino acid for F18 that is conservative with respect to S is F18T, F18C, F18Y, F18N, or F18Q, and the substitution of a different amino acid for F18 that is conservative with respect to V is F18G, F18A, F18V, F18L, F18I, F18M, F18W, or F18P.

6. The composition of claim 4, wherein the composition comprises a recombinant microorganism comprising the protein or the variant of the protein expressed by a foreign gene.

7. The composition of claim 6, wherein the microorganism is the genus Escherichia.

8. The composition of claim 4, wherein the protein or the variant thereof has 85% or more sequence identity with an amino acid sequence of SEQ ID NO: 1.

9. A method of reducing a concentration of a fluorine-containing compound in a sample, the method comprising:
contacting a haloacid dehalogenase hdl4a protein or a variant thereof with a sample comprising a fluorine-containing compound represented by Formula 1 or Formula 2, so as to reduce the concentration of the fluorine-containing compound in the sample,
wherein the variant comprises an amino acid alteration in an amino acid residue corresponding to position F18 of SEQ ID NO: 1:
C(R¹)(R²)(R³)(R⁴) <Formula 1>
(R⁵)(R⁶)(R⁷)C—[C(R¹¹)(R¹²)]n-C(R⁸)(R⁹)(R¹⁰), <Formula 2>
wherein, in Formula 1 and 2:
n is an integer from 0 to 10,
R¹, R², R³, and R⁴ are each independently fluorine (F), chlorine (Cl), bromine (Br), iodine (I), or hydrogen (H), wherein at least one of R¹, R², R³, or R⁴ is F; and
R⁵, R⁶, R⁷, R⁸, R⁹, R¹⁰, R¹¹, and R¹² are each independently F, Cl, Br, I, or H, wherein at least one of R⁵, R⁶, R⁷, R⁸, R⁹, R¹⁰, R¹¹, or R¹² is F.

10. The method of claim 9, wherein the amino acid alternation comprises substitution of D, S, or V or substitution of a different amino acid for F18 that is conservative with respect to D, S, or V, wherein the substitution of a different amino acid for F18 that is conservative with respect to D is F18E, the substitution of a different amino acid for F18 that is conservative with respect to S is F18T, F18C, F18Y, F18N, or F18Q, and the substitution of a different amino acid for F18 that is conservative with respect to V is F18G, F18A, F18V, F18L, F18I, F18M, F18W, or F18P.

11. The method of claim 9, wherein the protein or the variant thereof has 85% or more sequence identity with an amino acid sequence of SEQ ID NO: 1.

12. The method of claim 9, wherein the hdl4a protein or the variant hdl4a protein is in a recombinant microorganism that expresses the hdl4a protein or the variant hdl4a protein.

13. The method of claim 12, wherein the hdl4a protein or the variant hdl4a protein is contacted with the sample by culturing the microorganism with the sample.

14. The method of claim 13, wherein the microorganism is Escherichia.

15. A method of producing a microorganism having increased ability to remove a fluorine-containing compound in a sample, the method comprising:
introducing into a microorganism a gene that encodes a haloacid dehalogenase hdl4a protein or an hdl4a variant protein.

16. The method of claim 15, wherein the microorganism is Escherichia.

17. A variant haloacid dehalogenase hdl4a protein comprising an amino acid alteration in an amino acid residue corresponding to position F18 of SEQ ID NO: 1.

18. The variant of claim 17, wherein the amino acid alteration comprises substitution of D, S, or V or substitution of a different amino acid for F18 that is conservative with respect to D, S, or V, wherein the substitution of a different amino acid for F18 that is conservative with respect to D is F18E, the substitution of a different amino acid for F18 that is conservative with respect to S is F18T, F18C, F18Y, F18N, or F18Q, and the substitution of a different amino acid for F18 that is conservative with respect to V is F18G, F18A, F18V, F18L, F18I, F18M, F18W, or F18P.

19. The variant of claim 17, wherein the variant hdl4a protein has 85% or more sequence identity with an amino acid sequence of SEQ ID NO: 1.

20. A polynucleotide encoding the variant haloacid dehalogenase hdl4a protein of claim 19.
